(19) United States
(12) Patent Application Publication
(10) Pub. No.: US 2021/0121121 A1
(43) Pub. Date: Apr. 29, 2021

(54) PARKINSON?S DISEASE TREATMENT ADJUSTMENT AND REHABILITATION THERAPY BASED ON ANALYSIS OF ADAPTIVE GAMES

(71) Applicant: International Business Machines Corporation, Armonk, NY (US)

(72) Inventors: Celia Cintas, Nairobi (KE); Aisha Walcott, Nairobi (KE); Lucile Ter-Minassian, Nairobi (KE); Komminist Weldernariam, Ottawa (CA)

(21) Appl. No.: 16/665,445

(22) Filed: Oct. 28, 2019

(51) Int. Cl. A61B 5/00; G16H 20/30; G16H 20/70; A61B 5/11; G06N 3/02

(52) U.S. Cl. CPC A61B 5/4082; G16H 20/30; G06N 3/02; A61B 5/1116; G16H 20/70

(57) ABSTRACT

A Parkinson's disease treatment method, system, and computer program product, including administering a game to a user, monitoring a Parkinson's disease sign and a Parkinson's disease symptom based on a result of the user playing the game, adjusting treatment of the user depending on the Parkinson's sign and the Parkinson's symptom, and suggesting a tailored rehabilitation exercise for the user based on the treatment.

### BACKGROUND

The present invention relates generally to a Parkinson's disease treatment method, and more particularly, but not by way of limitation, to a system, method, and computer program product for improving Parkinson's disease treatment adjustment and rehabilitation therapy based on analysis of adaptive serious games.

Parkinson's disease is the second most common age-related neurodegenerative disorder. An estimated 7 to 10 million people worldwide have Parkinson's disease. It causes significant impairment and social difficulties. The disease's symptoms start gradually, sometimes starting with a barely noticeable tremor, stiffness or slowing of movement. Early symptoms may also be related to facial expression, posture and speech. Yet, these early signs may be mild and go unnoticed.

Although Parkinson's disease (PD) cannot be cured, Levedopa-based medications (i.e., a chemical compound) can significantly improve the patient's symptoms.

The adjustment of the medication is a challenge for clinicians. A clinician administers medications both seeking immediate benefits but also calculating the long-term effects of the medications on the course of the disease. Good medication response occurs within the therapeutic window and outside the window, and one might get motor fluctuations (not enough medication) or dyskinesia's (too much medication). “Off” periods and dyskinesia's are more frequent and severe as the disease progresses. This attempts to anticipate the effects of the drugs on the course of the disease that has created the most uncertainty and controversy in the field of Parkinson's disease (PD) therapeutics.

Indeed, in the conventional techniques, the dosage must prevent the patient from side-effects as much as possible. The side-effects of Levedopa are almost systematic and are always very impairing for the patient. The dosage and frequency depend on multiple symptoms. Yet, symptoms vary within a time-of-day (a week, and within a day). They depend on factors such as emotions, fatigue, activity, decay of the medication. As a consequence, it is difficult to judge a patient's symptoms based on a short, for example 30 minutes-to-one-hour, consultation only. The clinician has to take into account the initial honeymoon period when Levedopa almost completely reverses the signs and symptoms. And, at all times, the neurologist must disentangle symptomatic drug effects, long-term adverse effects and disease progression

Occasionally, a doctor may suggest surgery to regulate certain regions of one's brain and improve one's symptoms. Deep brain stimulation (DBS) is a surgery to implant a device that sends electrical signals to brain areas responsible for body movement. DBS can help reduce the symptoms caused by Parkinson's disease, but does not prevent them. Similarly, continuous care post-surgery is a key to improving the patient's life. As the disease keeps on progressing, regular check-ups are needed with the neurologist/neurosurgeon when PD symptoms intensity are assessed. From his/her observations, the clinician adjusts the parameters of the device (intensity, tension, plots of the electrodes, etc.).

Even when people with PD have access to a rehabilitation program with quality and qualified professionals, such a program may be long and may become unpleasant. In some cases, these individuals may present lack of motivation during the program due to its degree of repeatability that causes fatigues due to its tenuous nature. In addition, cases of depression and dementia arising from PD can withdraw these individuals from the special care routine. The use of games has proved to be a good way to fight lack of motivation that results from repetitive exercises on therapy programs.

While multiple prior techniques are based on intelligent wearables equipped with sensors to detect tremors, stiffness or slowed body movements, there is little work done to monitor speech abilities, facial expression and hand stiffness for PD treatment adjustment. Specifically, the conventional techniques do not cover aspects such as any type of PD treatment adjustment issue, both chemical and surgical and the use of adaptive serious games for symptoms assessment (not only rehabilitation). Also, the conventional techniques do not involve a method to customize the games according to the patient's impairment and his/her agenda. PD patients have the state of their disease assessed by their neurologist every 3 to 6 months. But, this assessment is qualitative and subjective (depends on the neurologist's judgment). This cannot be scalable as symptoms vary during the day, and between days. Thus, there is a lot of noise and having a single assessment is not significant.

Thus, there is a need in the field of continuous care for Parkinson patients for a new technique to augment a treatment adjustment and rehabilitation exercise plan, based on the patient's PD signs and his/her social/professional agenda.

### SUMMARY

Based on the above challenges and drawbacks in the conventional techniques, the inventors have recognized a solution that goes beyond looking at tremors/bradykinesia only, and monitors the full spectrum of PD symptoms, so as to improve continuous care for PD patients and ultimately ease the patient's daily life and social integration.

In an exemplary embodiment, the present invention provides a computer-implemented Parkinson's disease treatment method, the method including administering a game to a user, monitoring a Parkinson's disease sign and a Parkinson's disease symptom based on a result of the user playing the game, adjusting treatment of the user depending on the Parkinson's sign and the Parkinson's symptom, and suggesting a tailored rehabilitation exercise for the user based on the treatment.

One or more other exemplary embodiments include a computer program product and a system, based on the method described above.

Other details and embodiments of the invention will be described below, so that the present contribution to the art can be better appreciated. Nonetheless, the invention is not limited in its application to such details, phraseology, terminology, illustrations and/or arrangements set forth in the description or shown in the drawings. Rather, the invention is capable of embodiments in addition to those described and of being practiced and carried out in various ways and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

Aspects of the invention will be better understood from the following detailed description of the exemplary embodiments of the invention with reference to the drawings, in which:

FIG. 1 exemplarily shows a high-level flow chart for a Parkinson's disease treatment method 100 according to an embodiment of the present invention;

FIG. 2 exemplarily depicts a detailed flow chart of method 100 according to an embodiment of the present invention;

FIG. 3 exemplarily depicts a use case according to an embodiment of the present invention;

FIG. 4 exemplarily depicts a block diagram illustrating components of a computing system for intelligently monitoring Parkinson's disease signs and symptoms using games to adjust treatment depending on the symptoms and suggest tailored rehabilitation exercises for a patient according to an embodiment of the present invention;

FIG. 5 exemplarily depicts generating exercises according to an embodiment of the present invention;

FIG. 6 depicts a cloud-computing node 10 according to an embodiment of the present invention;

FIG. 7 depicts a cloud-computing environment 50 according to an embodiment of the present invention; and

FIG. 8 depicts abstraction model layers according to an embodiment of the present invention.

### DETAILED DESCRIPTION

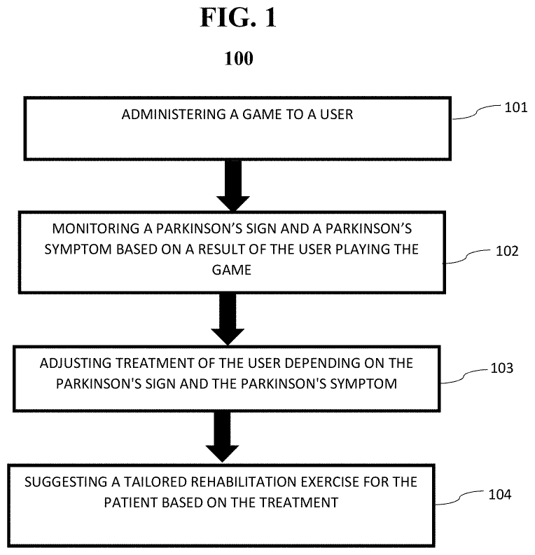

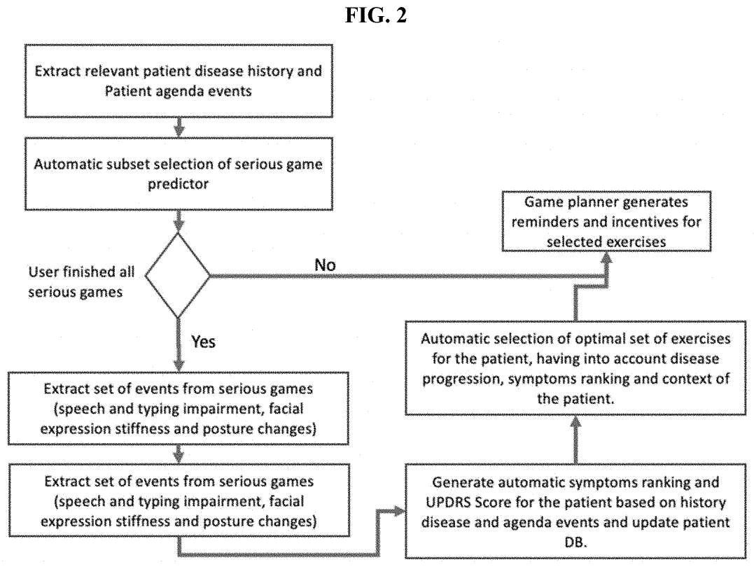

The invention will now be described with reference to FIGS. 1-8, in which like reference numerals refer to like parts throughout. It is emphasized that, according to common practice, the various features of the drawings are not necessarily to scale. On the contrary, the dimensions of the various features can be arbitrarily expanded or reduced for clarity.

By way of introduction of the example depicted in FIG. 1, an embodiment of a Parkinson's disease treatment method 100 according to the present invention can include various steps for determining a Parkinson's disease treatment score(s) for a location based on received valuation assessments from the crowdsourced users and/or devices.

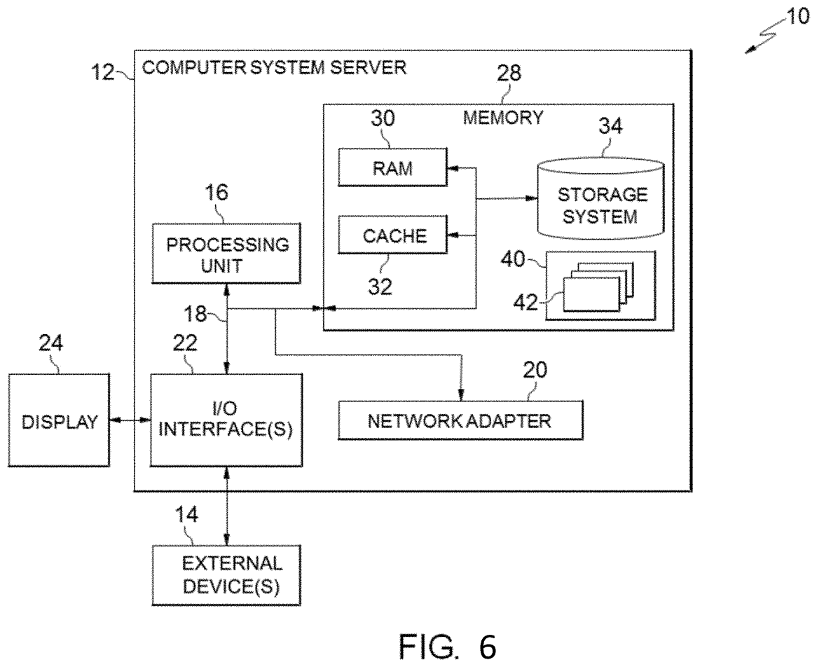

By way of introduction of the example depicted in FIG. 6, one or more computers of a computer system 12 according to an embodiment of the present invention can include a memory 28 having instructions stored in a storage system to perform the steps of FIG. 1.

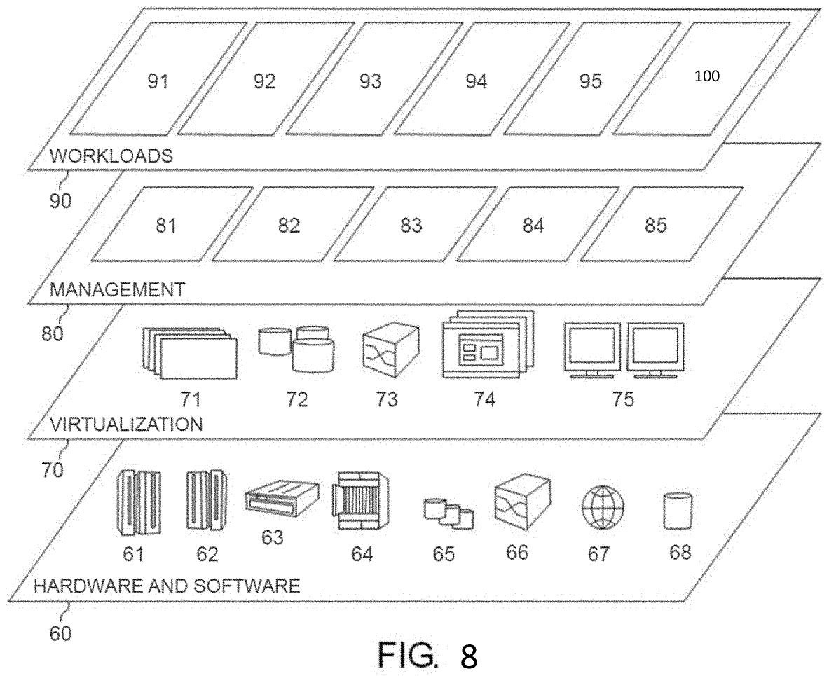

Although one or more embodiments may be implemented in a cloud environment 50 (e.g., FIG. 8), it is nonetheless understood that the present invention can be implemented outside of the cloud environment.

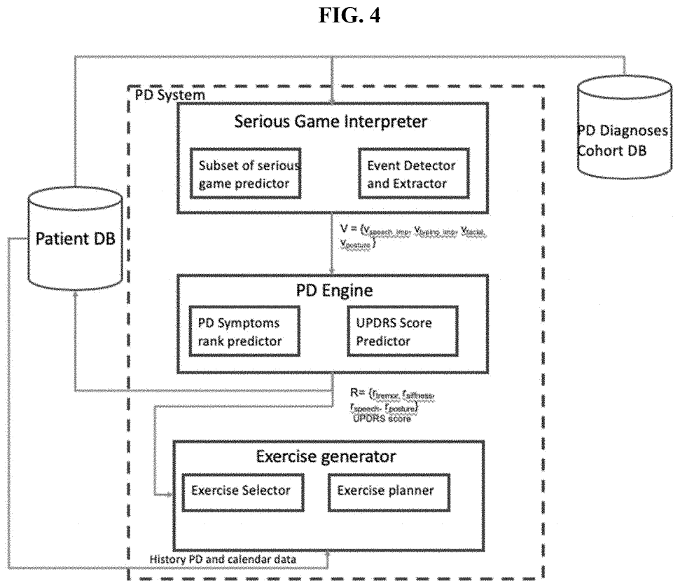

With reference to FIGS. 1-5 and as discussed later in more detail, in step 101, a game (for example, a serious game which is designed to assess various aspects of PD signs and symptoms in performing one or more tasks that may be affected/impaired by the symptoms) is administered to a user. For example, a “serious game” may be some sort of game which requires a patent to perform a task (i.e., a physical task, mental task, etc.). In step 102, a Parkinson's disease sign and a Parkinson's disease symptom of Parkinson's disease (PD) are monitored based on a result of the user playing the game. In step 103, treatment of the user is adjusted depending on the Parkinson's sign and the Parkinson's symptom. And, in step 104, a tailored rehabilitation exercise is suggested for the patient based on the treatment. FIG. 4 exemplarily depicts a system architecture to implement the method 100.

More specifically, the method 100 may intelligently monitor Parkinson's signs using adaptive serious games for symptoms assessment on user computing devices to adjust treatment depending on the symptoms and suggest tailored rehabilitation exercises for a patient, based on the patient's impairment, his/her agenda and treatment progress.

In one or more embodiments, performing an assessment of PD signs using games prescribed by the clinician games may be on patient's computing and/or communication devices (e.g., PC, notebooks, mobile phone, sensors, etc.). The games may be combined with motor neuronal tests performed by the neurologist during the usual check-up consultations. The games may be automatically generated by a deep neural network model trained using historical (clinician) games given to the patient, historical performance of these games (interactions and responses), historical motor neuronal tests, patient profile, current and predicted patient conditions and context, etc. The games to track PD symptoms may be done using models trained based on data collection from patient's keyboard interactions, microphone and camera usages. The method 100 using the games further includes assessing multiple symptoms, comprising tremors, stiffness, facial expression impairment, speech impairment, and/or posture. The games may be a tongue twister, the user preparing drawings, typing, etc. In one embodiment, scoring the games is based on symptom trajectories. The games scores will be further used to score PD symptoms.

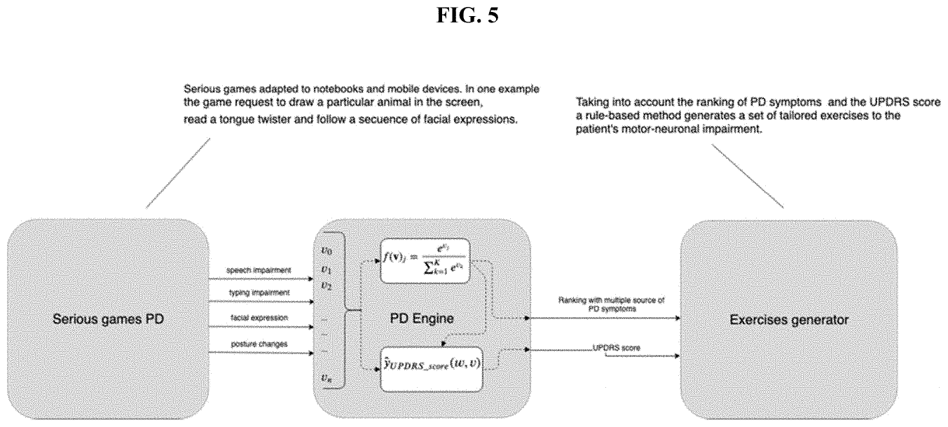

In one or more embodiments, such as shown in FIG. 5, the methods 100 model effects of PD on game abilities and computes game scores into PD symptoms scores. The modelling may involve the impairment caused by PD on typing abilities, mouse movements, speech, sitting posture and facial expression, by using one or more machine learning algorithms (e.g., trained deep learning deep learning models) for synthesizing, tracking and scoring changes in speech, postural, facial expression, movements of body parts while doing certain activities such as writing, typing, playing piano, etc. The evolution and trajectories of these changes over period of time can be modelled using sequence mining and time series algorithms. The models will take as an input the serious games scores and calculate each patient's Unified Parkinson's disease Rating Scale (UPDRS) score (S_{UPDRS}).

By way of example implementation, the PD Engine takes as an input a tuple v={v_{speech_imp}, v_{typing_imp}, v_{facial}, vₚₒₛₜᵤᵣₑ}, where, the v_{speech_imp}, score is calculated from the tongue twister serious game. The v_{typing_imp} is a score calculated from the keyboard pattern or drawing pattern in mobile devices. v_{facial} and vₚₒₛₜᵤᵣₑ are estimated from camera interactive games. All the values in the tuple are normalized [0, 1]. The method 100 uses a normalized exponential function, to assign a probability to a type of source PD symptoms using V as the vector with features corresponding to the analyzed patient (e.g., speech, tremors, stiffness, etc.), the output of this function is a K-dimensional vector (K will be the number of sources of symptoms) with values between [0, 1], all the entries add 1. The method 100 keeps the highest value as the most important symptom of PD.

It is noted that {v_{speech_imp}, v_{typing_imp}, v_{facial}, vₚₒₛₜᵤᵣₑ} are examples and the input tuple can include more or less values.

The UPDRS score is a value from [0, 199] and the score is generated from input values from V (highly correlated with usual provider questioners) and passed to a tuned weighted sum

$${S}_{UPDRS} = {\sum }_{j = 0}^{n} {w}_{j} {v}_{j} \qquad ( 1 )$$

In equation (1) for the UPDRS score, “j” is different aspects to assess such as: mentation, mood, and behavior, activities of daily living, motor examination, complications of therapy, etc. And, “v” is the input tuple.

In one embodiment, the method of suggesting therapeutic exercises and execution plans is based on PD symptoms scores, patient's schedule, and disease history. The method 100 in step 104 may suggest the therapeutic exercises from the ranking of PD symptoms and the overall score S_{UPDRS} a number of exercises tailored to the patient's motor-neuronal impairment, to rehabilitate some motor skills. The execution of the therapeutic exercises' plans may be used to further optimize scheduling of the patient serious games according to treatment, agenda, context, and medical/disease history.

The exercise suggestion takes as inputs the ranking of PD symptoms R={rₜᵣₑₘₒᵣ=0.9, r_{stiffness}=0.7, r_{speech}=0.5, rₚₒₛₜᵤᵣₑ=0.5} as generated by the PD Engine module, the overall score S_{UPDRS}, the patient's agenda, the data aggregated on the history of the disease progression, including past distribution of symptoms, etc.

The method 100 may optimize a calendar entry for therapy based on the inputs listed above to suggest the best exercises and time-of-day during the next few days (i.e., 10, 20, 30 days, etc.) for the patient's therapy that maximizes his/her time and helps efficiently with his/her therapy progress. The method 100 further estimates or predicts the outcome of the therapy progression based on ongoing and historical exercises. In an exemplary implementation, the method 100 may train a decision tree to provide the best subset of exercises or interventions. For instance, the set of interventions X could be X={Balance exercises with a chiropractor, Bi-monthly appointment with a speech pathologist, Gripping practices, Back, Wrist & Hand stretching exercises}.

In an additional embodiment, suggesting games for new symptom assessment is based on training one or more machine learning models using historical data including historical PD symptoms scores, treatment adjustment and therapeutic exercises. The outputs from the inventive models on therapeutic exercises, treatment adjustment and assessed PD symptoms are eventually looped back into the patient's disease history and will be used as future inputs. The next set of games is calculated from {last PD scores, updated disease history with current treatment & last set of therapeutic exercises, agenda}. The information transits in a looped fashion, similarly to a system control in engineering, with serious games replacing the sensors.

The method 100 may also optimize a calendar entry for calculating the optimal serious games for data collection from the ranking of PD symptoms, for example R={rₜᵣₑₘₒᵣ=0.9, r_{stiffness}=0.7, r_{speech}=0.5, rₚₒₛₜᵤᵣₑ=0.5}, generated by the PD Engine, the patient's agenda, the data aggregated on the history of the disease progression: past distribution of symptoms, treatment schedule and dosage; etc.

The method 100 may use a decision tree to provide the best subset of serious games. Using as an input R and A, where A={scoreavailable_time, deltapublic_speech} is agenda scores regarding time availability and public presentations delta is time until the next presentations. Given a new profile I={R, A}, the method 100 trains a machine learning model (e.g., random forest method) to classify the type of serious game SG to perform based on the input I. In an implementation, training a random forest model is used to classify the type of game G First, the model takes the test features from the new profile I and use the rules of each randomly created decision tree to predict the outcome content G Second, it calculates the votes for each predicted G (e.g., how many trees predicted the same G?). Third, the algorithm considers the high voted predicted G as the final prediction from the random forest algorithm (Output: type of G selected).

It is noted that the agenda embodiment prevents the patient from not being able to do the serious games he/she's been told to do if his schedule does not allow him/her, enables the system to prioritize the serious games if the patient is busy, and takes into account the moments where the patient will be talking in public to adjust his speech/facial expression in advance—maybe by prescribing the patient a higher dose during a limited period before the speech.

In an embodiment, step 103 can be implemented with a similar algorithm as the one detailed in the previous embodiment.

In another embodiment, the method 100 may adapt one's device to one's impairment caused by PD. That is, the method 100 may adapt the computer mouse and the screen display to the patient's moto-neuronal impairment. The method 100 may further adapt the keyboard and screen's features according to the PD scores collected. This can be done with a Deep Learning model that takes as an input a vector with all the scores outputted from the PD engine and outputs a vector with the angle of the screen, the optimal mouse pace and may provide some trackpad shortcuts ideas to facilitate computer use for the patient.

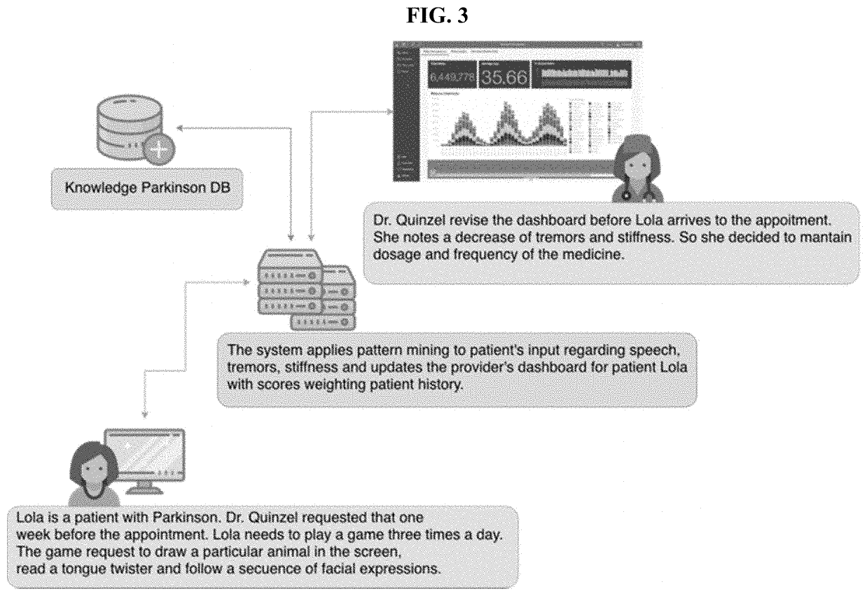

FIG. 3 exemplarily illustrates a use case for the invention. In the use case, Dr. Quinzel adopted the inventive technology of method 100 to improve the continuous care she provides to PD patients. The last time she saw her patient Lola, Dr. Quinzel had requested that she play a serious game three times a day and reads a tongue twister once a week. Before Lola arrives to her appointment, the doctor revises the dashboard that summarizes the serious games Lola has done over the past few months. She notices a decrease in stiffness and an increase in speech paralysis. Lola has updated her agenda for the next few months. She has a major conference coming up in two weeks and her speech must be smooth. The method 100 has calculated the optimal treatment schedule for the months to come and is waiting for Dr. Quinzel's approval, post discussion with Lola during the consultation, to increase frequency of intake of Levedopa from next week so to avoid any speech slowness during Lola's presentation. Furthermore, the method 100 suggests that Lola work on her enunciation with a bi-monthly appointment with a speech pathologist.

That is, the invention disclosed herein uses games (i.e., serious games) over mobile devices to extract patterns and to track symptoms without expensive or invasive sensors. The invention may adjust for rehabilitation exercises and treatment without taking into account events in a personal calendar and a context of the patient, only timestamps where the patient is at home or not. The invention aims to use adaptive games for symptoms assessment on user computing devices to adjust treatment depending on the symptoms, patient's impairment, his/her agenda (e.g. public speaking events, long trips, etc.), predictive treatment progress coupled with inferred context of the patient.

Exemplary Aspects, Using a Cloud Computing Environment

Although this detailed description includes an exemplary embodiment of the present invention in a cloud computing environment, it is to be understood that implementation of the teachings recited herein are not limited to such a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client circuits through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 6, a schematic of an example of a cloud computing node is shown. Cloud computing node 10 is only one example of a suitable node and is not intended to suggest any limitation as to the scope of use or functionality of embodiments of the invention described herein. Regardless, cloud computing node 10 is capable of being implemented and/or performing any of the functionality set forth herein.

Although cloud computing node 10 is depicted as a computer system/server 12, it is understood to be operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop circuits, multiprocessor systems, microprocessor-based systems, set top boxes, programmable consumer electronics, network PCs, minicomputer systems, mainframe computer systems, and distributed cloud computing environments that include any of the above systems or circuits, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing circuits that are linked through a communications network. In a distributed cloud computing environment, program modules may be located in both local and remote computer system storage media including memory storage circuits.

Referring now to FIG. 6, a computer system/server 12 is shown in the form of a general-purpose computing circuit. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further described below, memory 28 may include a computer program product storing one or program modules 42 comprising computer readable instructions configured to carry out one or more features of the present invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may be adapted for implementation in a networking environment. In some embodiments, program modules 42 are adapted to generally carry out one or more functions and/or methodologies of the present invention.

Computer system/server 12 may also communicate with one or more external devices 14 such as a keyboard, a pointing circuit, other peripherals, such as display 24, etc., and one or more components that facilitate interaction with computer system/server 12. Such communication can occur via Input/Output (I/O) interface 22, and/or any circuits (e.g., network card, modem, etc.) that enable computer system/server 12 to communicate with one or more other computing circuits. For example, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, circuit drivers, redundant processing units, external disk drive arrays, RAID systems, tape drives, and data archival storage systems, etc.

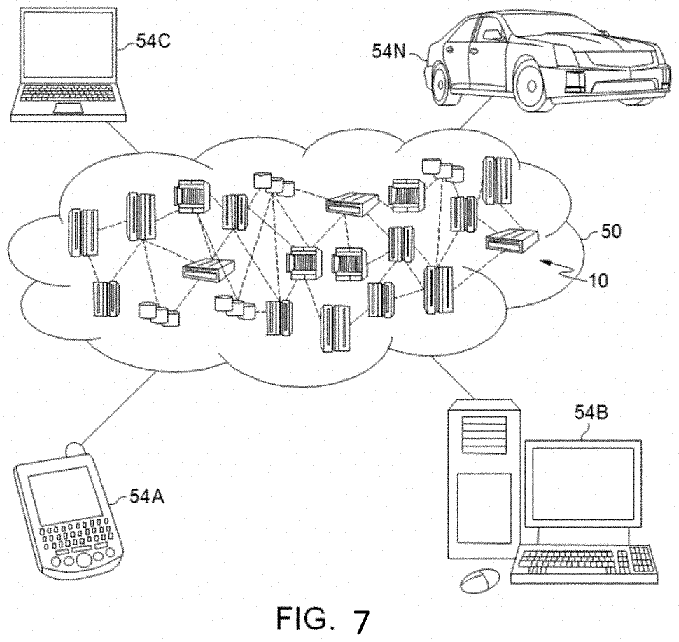

Referring now to FIG. 7, illustrative cloud computing environment 50 is depicted. As shown, cloud computing environment 50 comprises one or more cloud computing nodes 10 with which local computing circuits used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing circuit. It is understood that the types of computing circuits 54A-N shown in FIG. 7 are intended to be illustrative only and that computing nodes 10 and cloud computing environment 50 can communicate with any type of computerized circuit over any type of network and/or network addressable connection (e.g., using a web browser).

Referring now to FIG. 8, an exemplary set of functional abstraction layers provided by cloud computing environment 50 (FIG. 7) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage circuits 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and Parkinson's disease treatment method 100 in accordance with the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Further, Applicant's intent is to encompass the equivalents of all claim elements, and no amendment to any claim of the present application should be construed as a disclaimer of any interest in or right to an equivalent of any element or feature of the amended claim.

What is claimed is:

1. A computer-implemented Parkinson's disease treatment method, the method comprising:
administering a game to a user;
monitoring a Parkinson's disease sign and a Parkinson's disease symptom based on a result of the user playing the game;
adjusting treatment of the user depending on the Parkinson's sign and the Parkinson's symptom; and
suggesting a tailored rehabilitation exercise for the user based on the treatment.

2. The method of claim 1, wherein the Parkinson's sign and the Parkinson's symptom are automatically extracted from at least one of a speech and a facial expression of the user using non-invasive technology through the game.

3. The method of claim 1, wherein the game models effects of Parkinson's disease on at least one of a typing ability, a computer mouse movement, a speech, a sitting posture and a facial expression.

4. The method of claim 1, wherein the monitoring calculates a Unified Parkinson's disease Rating Scale (UPDRS) score for the user.

5. The method of claim 1, wherein the tailored rehabilitation exercise includes a different game for a second assessment based on analyzing a past Parkinson's sign and the Parkinson's symptom, a past treatment adjustment and a past therapeutic exercise.

6. The method of claim 1, wherein the tailored rehabilitation exercise includes modifying and controlling one or more display parameters of a device for the game.

7. The method of claim 1, wherein generating adaptive games with a deep neural network model trained using past games given to the patient, past performance of these games, past motor neuronal tests, patient profile, and current and predicted patient conditions and context.

8. A computer program product for Parkinson's disease treatment, the computer program product comprising a computer-readable storage medium having program instructions embodied therewith, the program instructions executable by a computer to cause the computer to perform:
administering a game to a user;
monitoring a Parkinson's disease sign and a Parkinson's disease symptom based on a result of the user playing the game;
adjusting treatment of the user depending on the Parkinson's sign and the Parkinson's symptom; and
suggesting a tailored rehabilitation exercise for the user based on the treatment.

9. The computer program product of claim 8, wherein the Parkinson's sign and the Parkinson's symptom are automatically extracted from at least one of a speech and a facial expression of the user using non-invasive technology through the game.

10. The computer program product of claim 8, wherein the game models effects of Parkinson's disease on at least one of a typing ability, a computer mouse movement, a speech, a sitting posture and a facial expression.

11. The computer program product of claim 8, wherein the monitoring calculates a Unified Parkinson's disease Rating Scale (UPDRS) score for the user.

12. The computer program product of claim 8, wherein the tailored rehabilitation exercise includes a different game for a second assessment based on analyzing a past Parkinson's sign and the Parkinson's symptom, a past treatment adjustment and a past therapeutic exercise.

13. The computer program product of claim 8, wherein the tailored rehabilitation exercise includes adapting a display parameter of a device for the game.

14. A Parkinson's disease treatment system, the system comprising:
a processor; and
a memory, the memory storing instructions to cause the processor to perform:
administering a game to a user,
monitoring a Parkinson's disease sign and a Parkinson's disease symptom based on a result of the user playing the game;
adjusting treatment of the user depending on the Parkinson's sign and the Parkinson's symptom; and
suggesting a tailored rehabilitation exercise for the user based on the treatment.

15. The system of claim 14, wherein the Parkinson's sign and the Parkinson's symptom are automatically extracted from at least one of a speech and a facial expression of the user using non-invasive technology through the game.

16. The system of claim 14, wherein the game models effects of Parkinson's disease on at least one of a typing ability, a computer mouse movement, a speech, a sitting posture and a facial expression.

17. The system of claim 14, wherein the monitoring calculates a Unified Parkinson's disease Rating Scale (UPDRS) score for the user.

18. The system of claim 14, wherein the tailored rehabilitation exercise includes a different game for a second assessment based on analyzing a past Parkinson's sign and the Parkinson's symptom, a past treatment adjustment and a past therapeutic exercise.

19. The system of claim 14, wherein the tailored rehabilitation exercise includes modifying and controlling one or more a display parameters of a device for the game.

20. The method of claim 14, wherein generating adaptive games with a deep neural network model trained using past games given to the patient, past performance of these games, past motor neuronal tests, patient profile, and current and predicted patient conditions and context.
